(19) United States
(12) Patent Application Publication
(10) Pub. No.: US 2013/0324521 A1
(43) Pub. Date: Dec. 5, 2013

(54) IMPLANT FOR TREATING OR PREVENTING AN ANEURYSM

(71) Applicant: Reza Ghotbi, Munchen (DE)

(72) Inventor: Reza Ghotbi, Munchen (DE)

(21) Appl. No.: 13/989,674

(22) PCT Filed: Nov. 24, 2011
(86) PCT No.: PCT/EP11/05918

§ 371 Date: Aug. 7, 2013

(30) Foreign Application Priority Data
Nov. 26, 2010 (DE) 20 2010 015 929.6
Feb. 24, 2011 (DE) 10 2011 012 224.9
Jul. 14, 2011 (DE) 10 2011 107 152.4

(51) Int. Cl. C07C 323/59; A61K 31/4709; C07D 401/14; A61K 31/5513; A61K 31/216; C07C 323/25; C07D 403/06; C07D 401/06; A61K 31/4545

(52) U.S. Cl. CPC C07C 323/59; C07D 401/06; A61K 31/4709; C07D 401/14; A61K 31/4545; A61K 31/216; C07C 323/25; C07D 403/06; A61K 31/5513
USPC 514/221; 546/157; 514/312; 546/93; 514/290; 560/9; 514/539; 560/10; 514/510; 540/570

(57) ABSTRACT

A medical product (1) introducible into a blood vessel (2) comprising a drug (3) for the treatment of an aneurysm and of arteriosclerotic diseases. The drug (3) is a farnesyltransferase inhibitor.

### FIELD OF THE INVENTION

The invention relates to a medical product that is insertable or implantable into a blood vessel and that contains at least one drug.

### BACKGROUND OF THE INVENTION

Aneurysms are permanent extensions of the diameter of blood vessels. Aneurysms occur pretty frequently. About 1 to 5% of the central European population are carrier of aneurysms. Mostly, aneurysms occur locally. But there are plenty of cases known in aneurysms frequently occur in different parts of the body. The multiple occurrence of aneurysms is also called Morbus Aneurysmaticus.

According to the size of the aneurysm and/or the localisation thereof in the patient's body, the occurrence of an aneurism may be life-threatening or at least cause pronounced symptoms. For example, an aneurysm may cause mechanical pressure to a nerve and thus result in paralysis or, when a rupture (fissuring or bursting) of the aneurysm occurs, the patient may bleed to death internally. Alternatively, a rupture can also first lead to a pulsatile haematoma that—at best—will be surrounded by a connective tissue-capsule within several weeks. In most cases, however, even upon a transient formation of a pulsatile haematoma, in the further course, a severe inner bleeding occurs which is not rarely causing the patient's death.

Furthermore an aneurysm may cause different dysfunctions of the body. For example, it may lead to a renal colic, an embolism or a thrombosis and may trigger an apoplectic stroke or a cardiac infarction. Basically, dysfunctions of the body may be caused by a swelling of the aneurysm and, thereby, applies pressure on the surrounding tissue or by a constriction of the lumen of vessels, like blood- or lymphatic vessels, and may result in bleedings and cause blood clots, that—for their part—decrease the lumen of vessels like blood- or lymphatic vessels. The person skilled in the art is aware of a variety of secondary damages caused by bleedings, reduced perfusion, reduced lymphatic flow and mechanical irritations of nerves.

The danger coming from an aneurysm typically depends on its size and its localisation, wherein particularly the rupture of an intracranial aneurysm or an aortic aneurysm often results in the death of the patient. A rupture of an intracranial aneurysm may lead to a cerebral haemorrhage (e.g., subarachnoidal-bleeding), a rupture of an aortic aneurysm may, for example, lead to an intraperitoneal haematoma (haemaskos).

Clinically, it is discriminated between symptomatic, asymptomatic and already raptured aneurysms. Especially asymptomatic aneurysms are often tardily identified as an incidental finding. Typically aneurysms are today diagnosed by viewing (inspection), fingering (palpation), ultrasonic testing(s) (sonography/sonographies), diagnostic radiology, computer tomography (CT), angiography or magnetic resonance imaging (MRI).

Due to the danger induced by aneurysms, today, at least larger aneurysms are treated preventively. The limit value for an aneurysm to be treated depends on several individual parameters that remain reserved for medical professionals, mostly aneurysms are however treated when the diameter is approximately 50% enlarged compared to the diameter of a healthy vessel. Generally, the treatment may be carried out surgically or endovascularly.

The surgical treatment premises that the vessel initially gets exposed. Such an exposure is particularly risky with intracranial aneurysms because a craniotomy (exposed brain-surgery) has to be carried out. But also other surgeries on the exposed aorta are indeed dangerous due to the large blood amount. When the vessel has been exposed appropriately, the aneurysmal sac is mostly clamped by a clamp (clipping) and the aneurysm is thereby separated from the blood circulation. Alternatively or additionally, the aneurysm-wall may also be externally strengthened (wrapping). As, however, such surgical treatment is laborious, expensive and dangerous, today, an endovascular treatment of aneurysms is increasingly preferred.

With the endovascular treatment of aneurysms a hollow micro-catheter is typically led through the vascular system to the aneurysmatic area by an easily accessible vessel such as, for example, the inguinal artery and, there, so-called coils are positioned into the aneurysmatic sac. Herein, it is mostly exploited that the surfaces of the coils, as surface foreign to the body, cause a local thrombus-formation and, thereby, prevent the blood circulation in the aneurism. Alternatively a vessel may also be strengthened endovascularly by using a vessel-support (stent).

In general, however, the methods known in the state of the art have the disadvantage that thereby only the symptoms of an aneurysm that has already occurred are treated, whereas the cause for the formation of an aneurysm is not treated to avoid subsequent diseases such as the occurrence of further aneurysms.

After an endovascular elimination of an aneurysm by an endograft, an expansion can occur particularly at the so-called “aneurysmatic neck” that leads to a dysfunction of the reconstruction. The long-term success of the endovascular therapy is significantly limited by this problem.

Aneurysms are caused by numerous reasons, such as, for example, degenerative vascular diseases (e.g., arteriosclerosis), traumata, infections (e.g., rheumatic fever, syphilis, Lyme disease), inflammations, congenital connective-tissue-weakness, a weakness of endothelial cells, Marfan syndrome, Ehlers-Danlos-Syndrome, cardiac defect (e.g., right-left-shunt), the appearance of Kawasaki syndrome, by external injuries and/or by internal injuries. Particularly often, aneurysms are caused by arteriosclerosis.

Arteriosclerosis or atherosclerosis, a disease based on vascular ageing processes, may be accelerated by several risk factors such as smoking, high-cholesterol nutrition or diabetes and may lead to debris in the arteries, plaque. Upon reaching a certain size, a stenosis, a constriction of the artery, develops. In this case, the perfusion of the organs is decreased. Such constrictions may next to the balloon-dilatation also be treated by means of stent implantations or by bypasses. The implants are placed into the artery to maintain the artery open.

For the treatment of arteriosclerosis, in the field of vascular medicine, implants are used, in that the therapist provides the constricted area of the blood vessel with an implant (e.g., a stent or stentgraft), dilates those (balloon angioplasty, drug eluted balloon) or bridges the constricted area with a bypass of synthetic or a body's own material.

Within a medical treatment, percutaneous transluminal angioplasty, PTA, also known as balloon-dilatation, the stenosis that obstructs the artery is removed by a thin catheter that is typically made of synthetic material and which ending contains an inflatable part (balloon). This catheter is mostly introduced via an inguinal vessel. From there, it is introduced into the concerned vessel under supervision on a screen. Once arrived in the constricted area, the balloon is inflated and, thereby, pushes the plaque to the vascular wall. This practice is also known as balloon dilatation. After the removal of the balloon the blood can flow again through the vessel. The concerned organ is supplied by blood again.

In many patients, after a balloon-dilatation, the opened artery obstruates again within some months. To keep the vessel open for a longer period of time, the physician, in particular cases, implants a stent after a successful balloon dilatation. This intervention is called stent-implantation. Sometimes stents that deliver medical agents that are intended to prevent a re-closing of the vessel are implanted to the vascular wall. Thus, in many cases, a restenosis (re-closing) may be prevented or retarded.

If an endovascular method is less promising due to technical or morphological reasons, the stenose is bridged by a bypass of synthetics or body's own-vein.

The vascular respectively endovascular administration of drugs for the treatment of arteriosclerosis by means of drug eluted balloons and stents with drug-coated prostheses and endoprotheses, respectively, is applied as state of the art.

From EP2193813A2, a local treatment of blood vessel diseases with drug-coated implants is known, that, for example, are intended to retard or even prevent a restenosis. However, in practice, also here the achieved results are not satisfying like in the treatment of aneurysms.

Furthermore, it is known that the method of EP2193813A2 is used for the treatment of arteriosclerosis by using a cancer drug (Tepe G, Schmitmeier S, Speck U, Schnorr B, Kelsch B, Scheller B, The Journal of Cardiovascular Surgery, February 2010).

The oral administration of drugs however provokes numerous undesired side-effects that are not justifiable in the therapy.

Alternatively or additionally to the induction of aneurysms due to arteriosclerosis, aneurysms may also be induced by a degeneration of the vascular walls, in particular at the aneurysm neck.

With an increasing age, this degeneration of the vascular walls normally develops, in particular with people having vascular diseases such as, for example, Marfan syndrome. Furthermore, a degeneration of the vascular walls may be provoked by an increased occurrence of proteases and an increasing destruction of smooth muscle cells in the muscular layer. Especially with patients having a consecutive dilatation, the outcome after an endovascular therapy may obviously be a limited one because the tissue is affected by a proceeding degeneration. Complications like graft migration, dislocation, endoleaks with repeated interventions may be the consequence of this set of problems that has not been solved so far.

Therefore, there is a need to develop a product enabling treating an already existing aneurysm and also enabling preventing the formation or recurrence of an aneurysm. This is particularly relevant for patients having a consecutive dilatation of the aneurysm neck in the further course.

### DESCRIPTION OF THE INVENTION

One aspect of the present invention is to provide a product that enables a treatment for cardiovascular diseases, wherein, however, harmful side effects are reduced. Especially, a treatment and prevention of aneurysms and arteriosclerosis may be enabled. A recurrence of arteriosclerotic or aneurysmatic alterations may be reduced by this specific treatment.

According to the invention, medical product introducible into a blood vessel comprising a drug for the treatment of an aneurysm and/or arteriosclerotic diseases is provided. The drug is a farnesyltransferase inhibitor.

In a first aspect, the present invention refers to a medical product (1, 11) introducible into a blood vessel (2, 12) comprising a drug (3, 13) for the treatment or prevention of an aneurysm, characterised in that said drug (3, 13) is a farnesyltransferase inhibitor.

As used in the context of the invention, the term “aneurysm” may be understood in the broadest sense as a permanent dilatation of the diameter of blood vessels. Preferably, with an aneurysm, merely a local circumscribed part of the vessel dilated. But it may also occur in different parts of the body which is called Morbus Aneurysmaticus. The dilatations may for an aneurism have any shape and may, for example, be formed spindle- or sack-like. An aneurysm may be congenital or acquired.

In a preferred embodiment, the aneurysm is an Aneurysma verum an Aneurysma spurium, an Aneurysma dissecans or a hybrid thereof, in particular an Aneurysma verum.

An Aneurysma verum is typically characterised in that it develops inside the vascular wall, so that the whole vascular wall with all layers (Intima, Media, Adventitia) is dilated. Often, an Aneurysma verum is provoked by one or more degenerative vascular disease(s) (e.g., arteriosclerosis, Marfan syndrome, Ehlers-Danlos-Syndrome) and/or one ore several peripheral arterial occlusive diseases. Common risk-factors for the formation of aneurysms are, for example, hypertension, smoking, copper shortage, diabetes, alcoholism, hypercholesterolemia, and/or old age.

An Aneurysma spurium, that may also be designated as Aneurysma falsum, is typically characterised in that it develops outside of the vascular wall and, therefore, the vascular wall layers Intima and Media are harmed, whereas the Adventitia maintains essentially intact. This injury can typically be caused by a dull or a sharp trauma. An Aneurysma spurium may result in a leak of the vessel and/or in a pulsatile haematoma. The pulsatile haematoma can only be surrounded by displaced tissue or by a connective tissue capsule.

An Aneurysma dissecans typically bases on a dissection, wherein the blood infiltrates between the layers of the vascular wall and separates them from each other. Thereby, a second bloodstream (pseudolumen) may be created. In particular upon the occurrence of an Aneurysma dissecans, ischaemiae of the original vessel may occur due to contractions of the vascular wall musculature.

An aneurysm in the sense of the invention may occur at any blood vessel, preferably at blood vessels with a higher internal pressure, therefore, at an artery or an arteriole, more preferably at an artery. For example, an aneurysm may be an aneurysm of the aorta (aortic aneurysm), of the Arteria carotis, of an artery of the upper or lower limb, of the Arteria subclavia, of a renal artery, of a cerebral artery (cerebral aneurysm), of the Arteria iliaca or of a coronary vessel. An aortic aneurysm may be distinguished between an abdominal aortic aneurysm (AAA) and a thoracic aortic aneurysm (TAA). An aortic aneurysm may be amongst others an aneurysm of the Aorta thoracica, Aorta abdominalis or of the abdominothoracic aorta.

In a preferred embodiment, the aneurysm is an aneurysm at the aorta, at the Arteria carotis, at an artery of an upper or lower limb, the Arteria subclavia, a renal artery, an encephalic artery, at the Arteria iliaca or at a coronary artery.

The person skilled in the art knows numerous reasons for the development of an aneurysm. Therefore, an aneurysm may be caused by a degenerative vascular disease (e.g., arteriosclerosis), traumata, infections (e.g., rheumatic fever, syphilis, Lyme disease), inflammations, congenital connective-tissue-weakness (e.g., Marfan syndrome, Ehlers-Danlos-Syndrome), a weakness of endothelial cells, cardiac defects (e.g., right-left-shunt), the occurrence of the Kawasaki syndrome, by external injuries and/or by internal injuries. Preferably, in the sense of the present invention, the aneurysm is an aneurysm of an artery caused by a degeneration of the arterial wall.

In a preferred embodiment, the aneurysm is caused by a degeneration of a vascular wall, in particular wherein the vascular wall is the aortic wall.

A degeneration of the aorta, in particular in the area of the aneurysm neck, may be caused by an increased occurrence of proteases and an increasing destruction of smooth muscle cells in the muscularis layer leading to the degeneration of the respective segment with a consecutive dilatation. Herein, also apoptotic cell death in the respective tissue may occur. The dilatation may be age-related and/or may be provoked by a disease (e.g., arteriosclerosis, Marfan syndrome, Ehlers-Danlos-Syndrome).

An aneurysm may be stabile or labile. It may optionally be surrounded by a connective tissue capsule. An aneurysm may burst spontaneously or as a consequence of external impact (rupture). A rupture may exemplarily result in an intraperitoneal haematoma (haemaskos) and/or in a cerebral bleeding (e.g., subarachnoidal bleeding) that may cause a bleeding to death. An aneurysm localised in the brain may optionally cause pressure on a nerve and result in paralysis. Therefore, also a not-bursting, thus, intact aneurism may cause death by breathing arrest. Furthermore, a thrombotic or embolic occlusion of one or more vessel(s) may occur. Therefore, for example, an apoplectic stroke, a cardiac infarction or an embolism in another organ (e.g., a pulmonary or renal embolism) may occur. The symptomatology may be acute or chronic. The person skilled in the art knows that the reset forces in an aneurysm typically behave inversely proportional to its diameter, so that a large aneurysm is typically more labile. Preferably, an aneurism in the sense of the present invention is a larger aneurysm that is endovascularly accessible, thus, an aneurysm having a maximum diameter of more than 0.2 cm, more than 0.3 cm, more than 0.4 cm, more than 0.6 cm, more than 0.8 cm, more than 1 cm, more than 2 cm, more than 3 cm, more than 4 cm, more than 5 cm, more than 6 cm, more than 7 cm, more than 8 cm, more than 9 cm or more than 10 cm.

An aneurism in the sense of the invention may be symptomatic (e.g., dorsal pain, abdominal pain, renal colic or pancreatitis, paralysis, hemiplegia, deficiency of speech, other brain damage) or asymptomatic. Reactions with several symptoms are also possible.

The diagnosis may be performed by every technique known by the person skilled in the art. Therefore, the diagnosis may, for example, be performed by viewing (inspection), fingering (palpation), ultrasonic testing(s) (sonography/sonographies), diagnostic radiology, computer tomography (CT), angiography or magnetic resonance imaging (MRI).

According to the invention, the medical product of the present invention can not only be used for the treatment and the prevention of an aneurysm, but may also be used for the treatment and the prevention of arteriosclerosis. The treatment or prevention of arteriosclerosis may, in turn, prevent the occurrence of an aneurysm.

Therefore, a second aspect of the present invention refers to a medical product (1, 11) introducible into a blood vessel (2, 12) comprising a drug (3, 13) for the treatment or prevention of an arteriosclerotic disease, characterised in that the drug (3, 13) is a farnesyltransferase inhibitor.

In a preferred embodiment, medical product (11) is an angioplasty-balloon with which the diseased area in the blood vessel (12) is treatable with the drug (13), farnesyltransferase inhibitor.

According to another preferred embodiment, the medical product (1) is implantable inside the blood vessel (2), wherein the drug (3), farnesyltransferase inhibitor, is administrable by means of the medical product (1) at the blood vessel (2).

According to an even more preferred embodiment, the medical product (1) includes a stent, an endograft or an endoprosthesis.

Therefore, a vascular implant may, for example, be a stent, an endoprosthesis, an endograft or a prosthesis coated with FTI. A stent is essentially comparable with a small tube that is made of a mesh wire.

According to an even more preferred embodiment, the medical product (1) includes a barestent and/or a stentgraft.

A bare-stent typically comprises a rigid wire netting and/or mesh wire that has the ability to keep a blood vessel open mechanically. Furthermore, a bare-stent may have a synthetic coating inside or outside of its lumen that is permeable or impermeable for fluids. An endoprosthesis is a mesh wire-implant with an integrated synthetic coating. In some special variations of these embodiments, the medical product includes a stent, an endograft, an endoprosthesis or a vascular prosthesis. A stentgraft typically consists of a tissue that is mostly composed of metal. The person skilled in the art will have noticed that a stentgraft may also be coated and/or contain non-metallic components or, alternatively, even be made of non-metallic components. Typically, the tissue of a stentgraft is a rigid tissue or mesh that has the ability to keep a blood vessel open mechanically.

According to a further preferred embodiment, the medical product of the present invention may include an anti-proliferative agent in addition to the drug (3, 13).

According to a further preferred embodiment, the medical product of the present invention may include a retarding adjuvant in addition to the drug (3, 13).

According to a further preferred embodiment, the medical product of the present invention may be intended for refilling respectively recoating with the drug (3, 13).

The medical product may comprise a balloon or a comparable product for vessel-dilatation. The balloon my form one end of a catheter, e.g., a vessel-catheter. The balloon may be intended for the performance of a balloon dilatation such as in the context of a percutaneous transluminal angioplasty (PTA).

The balloon may be part of a drug-coated balloon (“drug eluting balloon”). The surface of the balloon may be completely or partially coated with the drug. The coating may comprise a carrier and/or other agents such as, for example, a (further) anti-proliferative or anti-inflammatory agent.

The medical product may comprise an implant. Implants are artificial products that support or replace a biological structure in its function. Additionally or alternatively, implants my function as storage and depot, respectively, for substances such as drugs, so that the substance is administered to the surrounding biological tissue after the positioning or placement of the implant.

Implants are often made of metal or synthetic material (polymer). The basic structure (e.g., a scaffold) of the implant may completely or partially consist of a bio-inert material, for example, of a metal like titanium. The implant may include other components additionally to said scaffold. Some special variants of implants comprise, for example, synthetic-coated metal structures.

The metal used for the implant may exemplarily comprise stainless steel, cobalt, chrome, the “shape-memory” material consisting of nickel and titanium nitinol, titanium or alloys comprising of these materials, exemplarily titanium- or cobalt/chrome alloys.

Material used for the implant may also comprise ceramic materials or polymeric materials such as polyethylene, polyurethane, polyester such as, e.g polylactids or silicone.

The implant may be a vascular implant such as a stent. In many cases, a stent equals a small tube which is introduced into a vessel or a comparable passage to maintain it open, thus, to prevent a vascular occlusion.

The diameter of a stent is mostly minimized before its use, for example, by folding or furling. After positioning at the desired area, the diameter of the stent is increased such as by means of an inflatable balloon. After enlargement, the stent pushes into the surrounding tissue such as, e.g., a vascular wall and remains on the on site. Stents may exemplarily be introduced by using endoscopy.

Stents may, e.g., comprise a meshed matrix of material like metal (mesh wire) or synthetic material. The matrix may be coated.

The implant may be a vascular prosthesis, for example, an endograft or an endoprosthesis. The endograft (stentgraft) is a prosthesis to be placed inside a vessel, an endoprosthesis replaces a part of a vessel. The vascular prosthesis may comprise a scaffold of a synthetic material like polyethyleneterephthalate (PET) or polytetrafluorethylene (PTFE), or may comprise a metal mash. Some particular embodiments of a vascular prosthesis comprise a mesh wire with a synthetic coating in respectively on which the drug as well as, where appropriate, other agents and/or retarding adjuvants such as wax or resin is/are included.

The medical product may comprise several layers, for example, an implant may comprise a metallic scaffold with a coating of at least one synthetic respectively polymeric material such as, e.g., one or more of the aforementioned materials. A coating is generally understood in that the implant on a basic structure (a scaffold, e.g., a metallic mash) comprises a layer of a certain thickness of a particular material. The material may comprise a carrier material such as a polymer, the drug, another agent or several other agents and/or a retarding material, which retards the delivering of the drug and/or the further agents. The carrier material, more general the coating material overall, may be porose and thereby absorb respectively comprise the drug of the invention (and, where appropriate, further agents such as, e.g., inflammation inhibitors).

The medical product may be coated to absorb and retardedly unleash the drug in an appropriate manner. Herein, it may be a coating with a polymer (e.g., as carrier material).

The medical product may comprise at least one additional agent in addition to the drug itself. The additional agent may be intended to enhance the anti-proliferative effect (e.g., the additional agent may comprise paclitaxel). A further agent may be intended to prevent infections that could be provoked by the coating materials used; therefore, such an agent may comprise an anti-inflammatory agent. In addition to another anti-proliferative agent and/or an anti-inflammatory agent, an antibiotic agent, an antithrombotic agent, etc. may additionally or alternatively be present.

At some certain variations of the medical product of the invention the selfsame coating (e.g., comprising a polymer) for storage of the drug may be furnished together with the additional agents. Alternatively, several coatings made of different polymers may be considered, that are present side by side or one upon the other and wherein one coating has been furnished with the drug only and another coating has been furnished with the other agent only.

To avoid synthetic coatings, also other known approaches for providing micro-porose stent surfaces for the absorption and retarded delivery of a drug may be used.

Particular embodiments of the medical product of the invention have an additional, i.e., a further or second, coating to influence the retarded delivery of the drug in an appropriate way, e.g., to delay it. This additional coating may exemplarily comprise a coating material such as wax or resin that is preferably biocompatible. The additional coating may be present as additional, e.g., most external layer or may be present in combination with a carrier material, the drug and optionally further agents in common density ratio.

According to the invention, further a method for preparing a medical is provided, wherein a drug is added to the medical product. The drug is a farnesyltransferase inhibitor as described herein.

The method may comprise the preceding step, wherein a balloon or a basic structure of an implant is coated with a coating material. The coating material is intended for absorbing the drug and, for example, it is micro-porose in an appropriate way.

Particular embodiments of the method contain the subsequent step of depositing an additional coating to retard the delivery of the drug.

The drug may, e.g., be present in solution and it may be added onto the medical product by spraying respectively by a spraying-technology. In particular embodiments of the method, one or more coating materials are commonly solved with the drug and/or other agents in a solvent and are sprayed together on a basic structure such as a balloon or an implant scaffold.

The deposit by spraying may comprise several acts of spraying, e.g., a first (basic-) act of spraying and a second (ending-) act of spraying. Herein, the sprayed substance may have the same or different composition; e.g., may spray, in a first act of spraying, mainly or only the drug and, in a subsequent act of spraying, mainly or only a retarding adjuvant.

After spraying, the method may comprise a drying step that may comprise a passive or active drying, e.g., by air or another gas composition, and/or a warming of the medical product, e.g., by warming the environment and/or application of radiation such as infrared radiation. When several acts of spraying are performed, one or several acts of drying may optionally be provided in between.

In another aspect, the invention refers to a farnesyltransferase inhibitor for use in a method for the treatment or prevention of an aneurysm.

According to a preferred embodiment, the aneurysm is defined as above.

The treatment with a farnesyltransferase inhibitor may be carried out before, during or after the surgical or endovascular treatment of the aneurysm. Preferably, the treatment with a farnesyltransferase inhibitor is carried out during or after the surgical or endovascular treatment of the aneurysm.

The farnesyltransferase inhibitor may be administered locally or systemically. For example, it may be injected, delivered orally, subcutaneously, percutaneously or nasally or it may be present in a medical product in the sense of the invention.

In a preferred embodiment, the farnesyltransferase inhibitor is included in a medical product (1, 11) of the present invention.

This medical product may be used directly for treatment of an aneurysm. Alternatively or additionally, the surgical or endovascular treatment of the aneurysm may be carried out at first and, subsequently, a second medical product containing the farnesyltransferase inhibitor may be used for after treatment. A medical product used for after treatment is preferably a balloon.

In an alternative preferred embodiment, the farnesyltransferase inhibitor is used for after treatment of an aneurysm.

As used herein, the term “after treatment” may be understood in the broadest sense as a treatment after the elimination of an acute danger. Typically, the after treatment is preventive to prevent a further occurrence of an aneurysm. The after treatment may, however, also serve for the faster healing and/or preventing or minimising a side effect of the treatment of an aneurysm. Therefore, for example, the danger of an infection may be minimised.

After treatment may be carried out with a medical product of the invention.

Alternatively, in the context of an after treatment according to the invention, the farnesyltransferase inhibitor may however be administered independently from the medical product of the invention. A local administration is particularly preferred, in particular by means of a balloon catheter. Alternatively, the farnesyltransferase inhibitor may also be intravenously administered.

As used herein, the term “farnesyltransferase inhibitor” may be understood in the broadest sense as a molecule that prevents the enzymatically catalysed transfer of a farnesyl residue to a substrate. Herein, the substrate that is farnesylated is typically a polypeptide of at least four amino acids in length. A polypeptide comprises preferably a linear strand of amino acids linked with each other by amide bonds. A polypeptide may be at least four, more than four, more than ten, more than 20, more than 30, more than 50, more than 100, more than 200, more than 300, more than 400, more than 500 or even more than 600 amino acids long. Typically, a polypeptide that includes more than one secondary structure element is called protein domain, a polypeptide that possesses a complex tertiary structure is called protein. However, a polypeptide may also be a branched polypeptide in which one or more amino acid(s) is/are conjugated to the side chains of other amino acids. The polypeptide may also possess posttranslational modifications. Numerous posttranslational modifications are well known to those skilled in the art and exemplarily comprise lipidation, phosphorylation, sulfatation, glycosylation, shortening (truncation), cyclisation of several amino acid moietys, cyclisation of the polypeptide string, oxidation, reduction, decarboxylation, acetylation, amidation, deamidation, development of a disulphide bridge, development of pyroglutamate, attachment of one or several amino acid(s), attachment of one or several cofactor(s) (e.g., biotinylation, conjugation of a heme group), complexation of metal ions, non-metal ions, peptides, small-molecular molecules and/or conjugation of iron-sulphur-clusters. Several other cofactors can also be linked such as, exemplarily, ATP, ADP, NAD⁺, NAD⁺H⁺, NADP⁺, NADPH⁺H⁺, metal ions, anions or lipids. A polypeptide that is enzymatically catalysed farnesylysed preferably includes a CAAX-sequence-motive, at which C represents a cysteine moiety, A an aliphatic amino acid moiety and X another amino acid moiety that is identified by the enzyme that catalyses the farnesylation.

As used herein, the enzymatically catalysed transfer of a farnesyl residue describes a biochemical reaction in which a farnesyl residue is transferred to a substrate, preferably a polypeptide. An enzyme that catalyses the transfer of a farnesyl residue to a substrate is called farnesyltransferase. In this case, typically, activated farnesole is transferred. Activated farnesole is preferably farnesyldiphosphate (farnesylpyrophosphate, FPP).

Preferably, the polypeptide that represents the substrate is farnesylated to a cysteine moiety. So a thiolester is generated. The terms “thiolester” and “thioester” are exchangeable and describe a R₁—CO—S—R₂ group, wherein a thiolester can also comprise the tautomeric form of the ester R₁—COH═S—R₂. Preferably, the cysteine moiety that may be farnesylated is localised near to the C-terminal ending of the protein. Particularly preferably, the cysteine moiety of a CAAX-sequence-motive is farnesylated, wherein C represents a cysteine moiety, A an aliphatic amino acid moiety and X another amino acid moiety that is identified by the enzyme that catalyses the farnesylation.

The enzyme that catalyses the farnesylation is preferably a farnesyltransferase (FTase), that represents a prenyltransferase with the enzyme-classification-number EC 2.5.1.X, more preferably EC 2.5.1.29, EC 2.5.1.58 or EC 2.5.1.59, even more preferably EC 2.5.1.29 or EC 2.5.1.58. The enzyme typically binds one or several zinc ion(s) (Zn²⁺). Geranylgeranyltransferase may also be effective as farnesyltransferase in the sense of the invention, because this enzyme is also able to farnesylate particular polypeptides.

Every substance or every molecular composition that is able to decelerate or to prevent the enzymatically catalysed farnesylation may be a farnesyltransferase inhibitor. Preferably, a deceleration of the farnesylation rate may be understood as a deceleration of more than 10%, more preferred of more than 25%, even more preferred of more than 50%, even more preferred of more than 75%, even more preferred of more than 80%, even more preferred of more than 90% and most preferred of more than 95% by the addition of the farnesyltransferase inhibitor in an suitable concentration at the site of action compared to a similar reaction environment without addition of the farnesyltransferase inhibitor. A suitable concentration of the farnesyltransferase inhibitor at the site of action may preferably be less than 10 mM, less than 1 mM, less than 100 μm, less than 10 μM, less than 1 μM, less than 100 nM, less than 10 nM, or less than 1 nM. The concentration at the site of action is meant to be the concentration of the farnesyltransferase inhibitor in the direct environment of the tissue to be treated. The concentration at the site of action may be considerably different from the systemic concentration of the farnesyltransferase inhibitor that is typically measured in the blood.

The farnesyltransferase inhibitor may be an antimetabolite such as, exemplarily, an analogue of farnesole, farnesylphosphate, farnesyldiphosphate or a substrate peptide. The farnesyltransferase inhibitor may also be a molecule with a different structure, that may bind into the binding pocket of the peptide substrate or the farnesyldiphosphate. Alternatively, the farnesyltransferase inhibitor may be an allosteric inhibitor.

The farnesyltransferase inhibitor may have any molecular structure. For example, it may be a peptidic agent, a peptidomimetic or a non-peptidic small-molecular agent. A peptidic agent mostly consists of a peptide. However, the peptide may be conjugated to other molecular structures such as, exemplarily, to an organic, biologically compatible polymer (e.g., polyethylene glycol (PEG), polyethylenimine (PEI), hydroxypropyl methacrylamide (HPMA), to a lipid, an alkyl moiety or to another polypeptide. A peptidomimetic is an agent which molecular structure mimics a peptide. A peptidomimetic may contain, for example, beta-amino acids (1 amino acids), gamma-amino acids (γ amino acids) or D-amino acids or it may be made out of these or out of a combination of several thereof. A peptidomimetic may also be conjugated to other molecular structures such as, exemplarily, an organic biologically compatible polymer. A peptidomimetic may also be a retro-inverse peptide. A small molecule agent is a molecule with a molecular weight of less than 1500 Da, preferably less than 1000 Da, even more preferably less than 500 Da. A small molecule agent may also be conjugated to other molecular structures such as, exemplarily, an organic biologically compatible polymer.

The farnesyltransferase inhibitor may, e.g., be R11577 (Zarnestra, Tipifarnib), SCH66336 (Lonafamib), FTI-277, GGTI-298, BMS-214664, L-778 or L-123. The agent R11577 is particularly preferred. R11577 is an imidazole-containing heterocyclic, non-peptidomimetic agent. R11577 is also known as Zarnestra and represents an accredited drug. For example, R11577 can provoke a prevention of the progression of cardiovascular diseases in animal experiments with an oral administration (Capell B C, Olive M, Erdos M R, Cao K, Faddah D A, Tavarez U L, Conneely K N, Qu X, San H, Ganesh S K, Chen X, Avallone H, Kolodgie F D, Virmani R, Nabel E G, Collins F S, Proc Natl Acad Sci USA, October 2008). Oral administration of drugs in animal experiments could prevent the progression of cardiovascular (arteriosclerotic) diseases by a systemic use of the farnesyltransferase inhibitor ZARNESTRA. This use is helpful for the treatment of cardiovascular diseases but it also has harmful side effects that are not justifiable.

Farnesyltransferase inhibitors are already used as accredited therapeutic agents (ZARNESTRA) in oncological therapy. They show a low toxicity and oral administration is tolerated well.

The farnesyltransferase inhibitor may be covalently or non-covalently bound to the surface of the medical product or may be attached to the surface. Furthermore the farnesyltransferase inhibitor may be embedded in the medical product. A covalent binding may result from a direct binding of the farnesyltransferase inhibitor to the surface of the medical product or it may be bound via a molecular linker. A linker may be any biologically compatible material such as, exemplarily, polyethylene glycol (PEG), a peptide linker, a protein, an alkyl linker, a HPMA linker or a PLGA linker. The linker may be biologically inert or it may be biologically degradable. The linker may be covalently or non-covalently linked to the farnesyltransferase inhibitor and/or to the medical product, for example, via an esterification, a maleimid linking or via an amide linking. A non-covalent binding may be the binding of the farnesyltransferase inhibitor itself or of a linker that is associated with the farnesyltransferase inhibitor to the surface of the medical product; this binding is imparted by ionic interaction, hydrogen bridges or complex-binding. Examples for a non-covalent binding are the interaction between a streptavidin-coated surface and a farnesyltransferase inhibitor that is conjugated with biotin or the interaction between a farnesyltransferase inhibitor that is connected to a histidine-tag (his-tag) and a nickel-NTA-conjugated surface. Furthermore a GST-tag or something similar may be used. The farnesyltransferase inhibitor may also be attached to the surface due to interactions such as, exemplarily, hydrophobic interactions. Furthermore, the farnesyltransferase inhibitor may be embedded into the medical product, especially when it offers porose or semi-porose features, thereby, offers porosities into which the farnesyltransferase inhibitor may be absorbed, or the farnesyltransferase inhibitor is infused into the medical product during its production. The medical product may also consist of a biologically degradable material that dissolves or degrades in the patient's body and then it delivers the farnesyltransferase inhibitor or may be coated with a biologically degradable material that dissolves or degrades in the patient's body and then it delivers the farnesyltransferase inhibitor. Such a biologically degradable material is for example polylactic acid (PA), glycolic acid (GA), polylactic-co-glycolic acid (PLGA), polycaprolactone, polyglycolide, poly-3-hydroxybutyrate or co-polymeres of two or more thereof. The farnesyltransferase inhibitor may also be bound as micro-particle and/or as nano-particle associated with the medical material.

The farnesyltransferase inhibitor may be present in any suitable pharmaceutical composition. The pharmaceutical composition may contain one or several adjuvant(s) and/or medium material such as, exemplarily, one or several solution(s) (e.g., water, dimethyl sulfoxide (DMSO), ethanol, vegetable oil, animal oil, mineral oil, paraffin oil), one or several surface-active substance(s) (e.g., sodium dodecyl sulphate (SDS)), one or several dye(s) (e.g., TiO₂, alimental colours, fluorescein, derivatives of fluorescein, Cy dyes, alexa fluor dyes, S dyes, rhodamine, quantum dots), one or several foaming agent(s), one or several vitamin(s), one or several salt(s) (e.g., sodium, potassium, magnesium, calcium or zinc salts), one or several thickening agent(s) (e.g., carboxymethylcellulose (CMC), polyethylene glycol (PEG), sorbitol), one or several wetting agent(s) (e.g., sorbitol, glycerol, mannitol, propylenglycol, polydextrose), one or several other enzyme(s), one or several preserving agent(s) (e.g., benzoic acid, methylparaben), one or several emulsifying agent(s), one or several support media, one or several releasing agent(s), one or several antioxidant agent(s), one or several herbage extraction(s), one or several organic biocompatible polymer(s) (e.g., hydroxypropyl methacrylamid (HPMA), polyethylenimine (PEI), carboxymethylcellulose (CMC), polyethylene glycol (PEG)), one or several incorporation mediator(s) (e.g., polyethylenimine (PEI), dimethyl sulfoxide (DMSO), cell-penetrating peptide(s) (CPP), protein transduction domain(s) (PTD), antimicrobial peptides), one ore several sweetening agent(s) (e.g., sucrose, acesulfame K, saccharine), one or several homoeopathic supplement(s) and/or one or several flavour(s).

The farnesyltransferase inhibitor may be administered locally or systemically. A systemic administration may, exemplarily, be an oral (e.g., as powder, tablet, pill, capsule, dragée, syrup or fluid), an intramuscular (e.g., via a syringe, cannula), an intravenous (e.g., via a syringe, cannula), an intraarterial (e.g., via a syringe, cannula), a subcutaneous (e.g., via a syringe, cannula, a reservoir (e.g., a PLGA-reservoir)), a percutaneous (e.g., as plaster, cream, ointment, lotion) or a nasal administration. A local administration may be an administration via the medical product. The medical product may contain the farnesyltransferase inhibitor wherein it is embedded in the material of the medical product, optionally in a pharmaceutical composition. The farnesyltransferase inhibitor may be present dissolved as fluid or syrup or as a dry substance. It may be dried or freeze-dried.

According to another aspect, the invention refers to a farnesyltransferase inhibitor for use in a method for the treatment or prevention of arteriosclerosis, wherein the farnesyltransferase inhibitor is included in a medical product (1, 11) corresponding to the invention.

In a preferred embodiment, the farnesyltransferase inhibitor is locally administered and the medical product (1, 11) is placed and/or fixed at the diseased area and said farnesyltransferase inhibitor is delivered at the diseased area.

As used herein, placing preferably means the introduction of the medical product into a vessel. It may stay there temporarily as it is, exemplarily, usually found for a balloon catheter or it may be fixed. The fixing includes, amongst others, the pressing on the vascular wall, the optional suture of the medical product into a tissue and/or the engraftment of the medical product into a tissue.

According to another preferred embodiment, the farnesyltransferase inhibitor is R11577, SCH66336, FTI-277, GGTI-298, BMS-214664, L-778 or L-123. Even more preferably, the farnesyltransferase inhibitor is R11577.

The farnesyltransferase inhibitor may be combined with other therapies such as, exemplarily, ultrasound treatment, radiotherapy (e.g., ultraviolet irradiation (UV-irradiation), X-ray irradiation, smooth X-ray irradiation, radioactive irradiation (e.g., α-radiation, β-radiation, γ-radiation), cosmic radiation), treatment with one or several inhibitors of proliferation (e.g., alkylant (cisplatin, carboplatin, satraplatin, oxaliplatin, mechlorethamine, cyclophosphamide, chlorambucil, ifosfamide), signal-transduction-inhibitor, vinca alkaloid (e.g., vincristine, vinblastine, vinorelbine, vindesin), taxane, podophyllotoxine, inhibitor of topoisomerase, anti-metabolite (e.g., methotrexat, dihydrofolate, 5-fluorouracil), antineoplastic agent). Furthermore, the farnesyltransferase inhibitor can also contain anti-inflammatoric agents (e.g., acetylsalicylic acid (ASS), benzydaminhydrochloride, bufexamac, diclofenac, diflunisal, flurbiprofen, ibuprofen, indomethacin, ketoprofen, mefenamine acid, metamizole, naproxen, oxyphenbutazone, phenylbutazone, phenazone, piroxicam, propyphenazone, salicylamide, tarenflurbil, tiaprofen acid, tenoxicam, tolfenamine acid, glucocorticoids, betamethasonvalerat, clobetasolpropionate, dexamethason, hydrocortisone, hydrocortisone acetate, prednicarbat, prednisolone, prednisone, triamcinolone acetonide, budenosid, fluticasone, montelukast, willow bark, camomile blossoms, calendula blossoms, arnica blossoms, roots of devil's claw, ash tree bark, poplar bark, goldenrod, horse chestnut, incense). The person skilled in the art will understand that also other pharmaceutically active agents may be associated with the medical product. The person skilled in the art will also understand that the above mentioned agents may also be directly connected to the medical product or via the spacer and/or may be linked to micro- or nanobeads.

The farnesyltransferase inhibitor may achieve the effect that a stenosis may be treated in a better way. Therefore, the vascular occlusion may be prevented in a better way and/or the side effects such as, exemplarily, the manifestation of an inflammation may be prevented, delayed or decreased. Therefore, the tendency towards inflammations may be reduced that, amongst others, results in a decreased formation or less secretion of inflammatory mediators (e.g., one or several prostaglandin(s), one or several thromboxane(s), one or several cytokines and/or one or several interleukin(s)). Furthermore, the tendency to a swelling, reddening and/or a manifestation of sensation of pain may be decreased. The farnesyltransferase inhibitor can also achieve that a stenosis may be prophylactically prevented in a better way or that at least the tendency towards a stenosis is reduced.

Arteriosclerosis is a pathological state caused by debris of body's own substances such as, exemplarily, blood-fat, thrombi, cholesterol, connective tissue, collagen, chalk, low density lipoprotein (LDL), high density lipoprotein (HDL) and/or proteoglycans on or in the vascular walls. Arteriosclerosis is also known as atherosclerosis, cardiovascular arteriosclerosis, cardiovascular atherosclerosis, cardiovascular plaques, colloquially also as calcification of the arteries. Arteriosclerosis can develop inside the intima and/or the media. Arteriosclerosis of the intima often occurs as a prosperity illness and is often nutrition related. However, arteriosclerosis of the media is often a calcinosis of the media that can for example be provoked by Mönckeberg's arteriosclerosis. Regarding Mönckeberg's arteriosclerosis, the blood vessels, as calcium reservoirs, harden in the medial layer of the vascular wall (tunica media), in particular of the mid-size vessels.

Arteriosclerosis also comprises the chronically developing focal modifications of the mesenchymal cells of the inner vascular wall (intima) and of the inner layers of the medial vascular wall (media). The modifications are also called plaques.

Furthermore, other factors such as, exemplarily, endothelial cells, smooth muscle cells, monocytes, macrophages, platelets, lipoproteins, growth factors and chemotactic factors (cytokines) may be involved in developing of arteriosclerosis.

Arteriosclerosis may result in a connective tissue-related hardening of the vessels. Furthermore an inadequate blood supply of a part of the body can occur. Ischemia, thrombosis, angina pectoris, cardiac infarction, apoplectic stroke and/or sudden death may be the consequences. The symptoms of arteriosclerosis are also called Arteriosclerotic Vascular Disease (ASVD).

By using anti-tumorous drugs, until now, mainly strategies that target on the increased proliferation frequency of cells in the vascular wall compared to healthy tissue have been pursued.

A reduction of the proliferation rate may, amongst others, be achieved with the help of inhibitors.

The invention also refers to a farnesyltransferase inhibitor for use in a method for the treatment of Hutchinson-Gilford progeria syndrome (HGPS), in particularl for the treatment or prevention of arteriosclerosis associated with HGPS.

In the context of the present invention, the successful use of farnesyltransferase inhibitors for the treatment of the rare genetic disease HGPS (Hutchinson-Gilford progeria syndrome) is highly interesting. This disease makes the affected children olden early and typically die at an early stage of a cardiac infarction respectively of a vascular caused apoplectic stroke within 20 years. Immunohistochemical analyses of this patients show an accumulation of the protein progerin in the cells.

The blockage of the farnesylation reduces the toxicity of progerin. The big improvement in this domain is the therapy with farnesyltransferase inhibitors that results in a complete normalization of the treated progeria cells. Progerin may play a significant role considering the development of arteriosclerosis.

Medical research has clarified that progerin plays a significant role considering the development of arteriosclerosis and it is also known that farnesyltransferase inhibitors reduce the toxicity of progerin. Based on this finding, a lot of drugs have been developed that contain as agent farnesyltransferase inhibitors (Olive M, Harten I, Mitchell R, Arterioscler Thromb Vase Biol. November 2012).

Analysis of patients with arteriosclerosis also show a significantly higher concentration of progerin in the coronary arteries of these patients. Therefore, there exist obvious hintsights for the fact that progerin also plays a significant role in the development of arteriosclerotic diseases.

So far, farnesyltransferase inhibitors have only been utilised for tumour treatment. A vascular, endovascular or systemic application for treatment of aneurysmatic as well as arteriosclerotic diseases has not taken place until now.

The aforementioned problem is solved according to an embodiment of the invention in that a medical product that is an implant, exemplarily a stent, a stentgraft, a vascular prosthesis or a balloon is introduced into a blood vessel for the treatment of aneurysms and arteriosclerotic diseases. The implant contains a drug that is a farnesyltransferase inhibitor. The inhibitor affects the aneurysmatic and arteriosclerotic disease and improves it.

In the treatment of an aneurysm and the arteriosclerosis, a completely new cellular target with normalisation of the cell function of the vascular wall is achieved by local administration of the farnesyltransferase inhibitors.

From these findings, the local vascular respectively endovascular administration of farnesyltransferase inhibitors in the form of balloons that are coated with them, stents, endografts respectively vascular prostheses is an effective therapeutic option. Thereby, the arteriosclerotic vascular lesion may be stabilised at the cell biological level in a way that the repeated occurrence of aneurysms as well as restenosis and progression of the diseases is effectively prevented.

### BRIEF DESCRIPTION OF THE FIGURES

Exemplary embodiments of the medical product of the invention are explained by the following drawings. It shows:

FIG. 1 a schematic 3D presentation of the medical product as a longitudinal profile through the blood vessel; and

FIG. 2 a schematic 2D presentation of the medical product as a longitudinal profile.

### DETAILED DESCRIPTION OF THE EXEMPLARY EMBODIMENTS

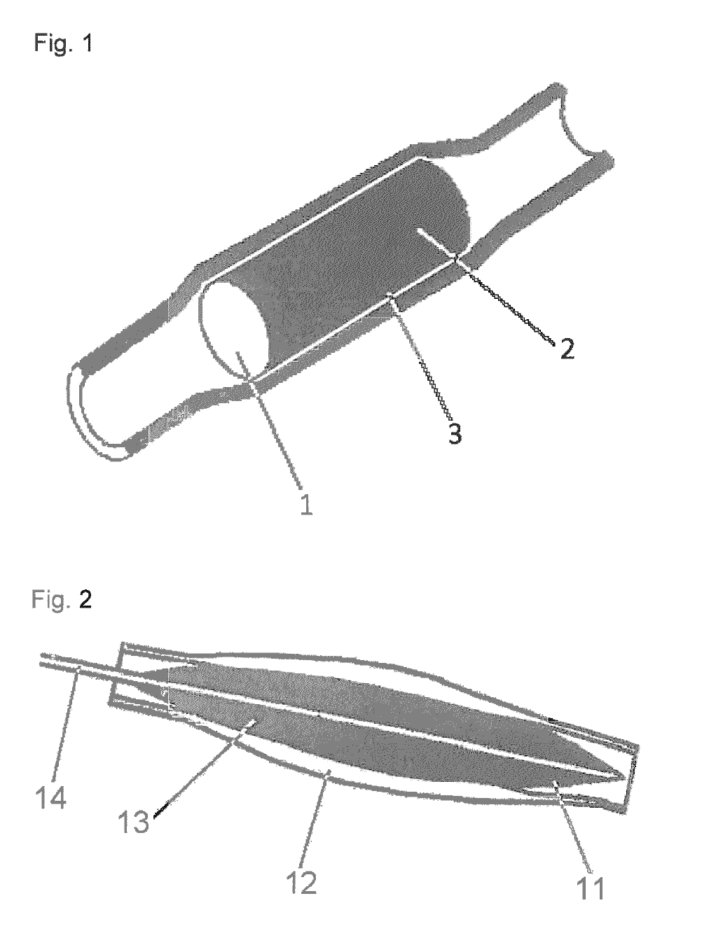

In an exemplary embodiment, the medical product 1 contains a drug 3 that is a farnesyltransferase inhibitor. As depicted in FIG. 1, the medical product 1 is introduced into a blood vessel 3 and administered to the aneurysmatically and/or arteriosclerotically diseased area. The drug 3 affects the aneurysmatic and/or arteriosclerotic vascular lesions and stabilises them.

Upon use at the bypass materials (PTFE, Dacron, vein), the farnesyltransferase inhibitors prevent the development of restenoses (hyperplasia of the intima).

The medical product 1 may be a stent, a vascular prosthesis, an endograft or another implant containing the drug 3.

The medical product 1 may be soaked with the drug 3 or coated with the drug 3 or connected therewith.

The stent 1 consists of a metal mesh that is coated with a micro-porous polymer-material. In the production of the stent 1, the hollow spaces of the material have been filled with a farnesyltransferase inhibitor as well as paclitaxel as another anti-proliferative agent by spraying or soaking. A wax as retarded adjuvant has been applied on the stent 1 (not explicitly emphasised in FIG. 1).

After the insertion into the vessel 2 and the anchoring (expansion) of the stent 1 at the area to be treated, the stent 1 prevents, together with the included agents, a (eventually repeated) vascular occlusion.

In other exemplifying embodiments, the farnesyltransferase inhibitor may be used eventually, depending on a specific disease entity, even without another anti-proliferative agent.

The type and the amount of the retarding adjuvant added may be chosen according to the specific case of application.

In another exemplifying embodiment, the medical product 11 is a balloon that forms one ending of a catheter 14. The balloon 11 contains the drug 13 and is introduced into the blood vessel 12 for therapeutic use and it is dilated at the aneurysmatically and/or arteriosclerotically diseased area (FIG. 2). So the stenosis is expanded and then the drug 13 affects the arteriosclerotic disease and provokes a healing.

The balloon 11 has been soaked with the drug 13 during the production. As a result, the balloon 11 is equipped with the farnesyltransferase inhibitor on its whole surface. At other performance examples the balloon is only partially equipped with the drug, for example at its middle part without being equipped with the drug at its proximal respectively distal segment (for example bordering respectively across from a catheter orifice).

Another agent such as, exemplarily, paclitaxel as anti-proliferative agent has been applied together with the farnesyltransferase inhibitor to the surface of the balloon 11. A retarding adjuvant is not provided because the therapeutically effective substances shall pass over into the tissue during the comparatively short period of time in which the balloon 11 reaches and dilates the area at the vessel 12 to be treated.

The invention further includes the following embodiments:
1. A medical product introducible into a blood vessel comprising a drug for the treatment of aneurysms and arteriosclerotic diseases, characterised in that the drug is a farnesyltransferase inhibitor.
2. The medical product according to embodiment 1, wherein said medical product is implantable into the blood vessel, wherein the drug, farnesyltransferase inhibitor, is administrable to said blood vessel by the medical product.
3. The medical product according to embodiment 1, wherein said medical product is an angioplasty-balloon with which the diseases area in the blood vessel is treatable by means of the drug, farnesyltransferase inhibitor.
4. The medical product according to embodiment 1 and 2 is an endograft.
5. The medical product according to embodiment 1 and 2 is a stent.
6. The medical product according to embodiment 1 and 2 is a prosthesis.
7. The medical product according to embodiment 1 and 2 is an endoprosthesis.
8. Use of a medical product for the treatment of aneurysms and arteriosclerosis according to embodiments 1, 2, 4, 5, 6 and 7, wherein said medical product is introduced into the blood vessel and placed and fixed at the diseased area, wherein the drug is administered onto the diseased area of the blood vessel.
9. Use of a medical product for the treatment of aneurysms and arteriosclerosis according to embodiments 1 and 3, wherein said medical product is an angioplasty-balloon with which the disease area in the blood vessel is treated.
10. Use of a medical product for the treatment of aneurysms and arteriosclerosis according to embodiments 1, 2, 4, 5, 6 and 7, wherein an implantable medical product is refilled respectively recoated with drug.

1. A medical product (1, 11) introducible into a blood vessel (2, 12) comprising a drug (3, 13) for the treatment or prevention of an aneurysm, characterised in that said drug (3, 13) is a farnesyltransferase inhibitor.

2. The medical product according to claim 1, wherein the aneurysm is caused by a degeneration of a vascular wall, in particular wherein said vascular wall is the aortic wall.

3. The medical product according to claim 1 or 2, wherein the aneurysm is an Aneurysma verum an Aneurysma spurium, an Aneurysma dissecans or a hybrid thereof, in particular wherein said aneurysm in an Aneurysma verum.

4. The medical product according to at least one of the claims 1 to 3, wherein the aneurysm is an aneurysm at the aorta, at the Arteria carotis, at an artery of an upper or lower limb, the Arteria subclavia, a renal artery, an encephalic artery, at the Arteria iliaca or at a coronary artery.

5. A medical product (1, 11) introducible into a blood vessel (2, 12) comprising a drug (3, 13) for the treatment or prevention of an arteriosclerotic disease, characterised in that the drug (3, 13) is a farnesyltransferase inhibitor.

6. The medical product according to at least one of the preceding claims, wherein said medical product (11) is an angioplasty-balloon with which the diseased area in the blood vessel (12) is treatable with the drug (13), farnesyltransferase inhibitor.

7. The medical product (1) according to at least one of the preceding claims, wherein said medical product (1) is implantable inside the blood vessel (2), wherein the drug (3), farnesyltransferase inhibitor, is administrable by means of the medical product (1) at the blood vessel (2).

8. The medical product (1) according to claim 7, wherein said medical product (1) includes a stent, an endograft or an endoprosthesis.

9. The medical product (1) according to claim 7, wherein said medical product (1) includes a barestent and/or a stentgraft.

10. The medical product according to at least one of the preceding claims, including an anti-proliferative agent in addition to the drug (3, 13).

11. The medical product according to at least one of the preceding claims, including a retarding adjuvant in addition to the drug (3, 13).

12. The medical product according to at least one of the preceding claims, wherein said medical product is intended for refilling respectively recoating with the drug (3, 13).

13. A farnesyltransferase inhibitor for use in a method for the treatment or prevention of an aneurysm.

14. The farnesyltransferase inhibitor according to claim 13, wherein the aneurysm is defined as in claims 2 to 4.

15. The farnesyltransferase inhibitor according to any one of claims 13 or 14, wherein said farnesyltransferase inhibitor is included in a medical product (1, 11) in accordance with at least one of claims 1 to 12.

16. The farnesyltransferase inhibitor according to one of claims 13 to 15, wherein said farnesyltransferase inhibitor is used for after treatment of an aneurysm.

17. A farnesyltransferase inhibitor for use in a method for the treatment or prevention of arteriosclerosis, wherein said farnesyltransferase inhibitor is included in a medical product (1, 11) according to at least one of the claims 1 to 12.

18. The farnesyltransferase inhibitor according to at least one of the claims 13 to 17, wherein the medical product (1, 11) is placed and/or fixed at the diseased area and said farnesyltransferase inhibitor is delivered at the diseased area.

19. The farnesyltransferase inhibitor according to one of claims 17 or 18, wherein the arteriosclerosis occurs in a patient with Hutchinson-Gilford Progeria Syndrome (HGPS).

20. A farnesyltransferase inhibitor for use in a method for the treatment of Hutchinson-Gilford Progeria Syndrome (HGPS).

21. The farnesyltransferase inhibitor according to at least one of the preceding claims, wherein said farnesyltransferase inhibitor is R11577, SCH66336, FTI-277, GGTI-298, BMS-214664, L-778 or L-123.
